(19) United States
(12) Patent Application Publication
(10) Pub. No.: US 2025/0213739 A1
(43) Pub. Date: Jul. 3, 2025

(54) STERILIZATION METHOD AND STERILIZATION SYSTEM

(71) Applicants: SHIMADZU CORPORATION, Kyoto-shi (JP); Shyld AI Inc., Santa Clara, CA (US)

(72) Inventors: Shin FUJITA, Kyoto-shi (JP); Tsunehiro INOUE, Kyoto-shi (JP); Shingo UNO, Kyoto-shi (JP); Naoya ISHIGAKI, Kyoto-shi (JP); Naoki WAKABAYASHI, Kyoto-shi (JP); Katsuhiko TOKUDA, Kyoto-shi (JP); Koji TOJO, Kyoto-shi (JP); Morteza NOSHAD, Fremont, CA (US)

(21) Appl. No.: 18/847,624

(22) PCT Filed: Mar. 16, 2023
(86) PCT No.: PCT/IB2023/000111

§ 371 Date: Sep. 16, 2024

Related U.S. Application Data
(60) Provisional application No. 63/320,828, filed on Mar. 17, 2022.

(51) Int. Cl. A61L 2/10; A61L 2/24

(52) U.S. Cl. CPC A61L 2/10; A61L 2/24; A61L 2202/14

(57) ABSTRACT

There is provided a highly flexible sterilization method that is also safe and optimal. This sterilization method includes sterilization area setting in order to set in advance a sterilization area within a predetermined area of human activity, peripheral area setting in order to set in advance peripheral areas that are adjacent to or in proximity to the sterilization area, irradiation mode deciding in order to decide an irradiation mode for irradiating sterilization electromagnetic waves onto the sterilization area based on peripheral area information acquired from the peripheral areas, and sterilization electromagnetic wave irradiating in order to irradiate sterilization electromagnetic waves onto the sterilization area in the irradiation mode.

### TECHNICAL FIELD

The present invention relates to a sterilization method and the like that sterilize a predetermined area using sterilization electromagnetic waves such as ultraviolet rays or the like.

### TECHNICAL BACKGROUND

Conventional UV sterilizers that are installed in rooms and facilities and the like irradiate surrounding areas indiscriminately with ultraviolet rays so as to disinfect the entire area reached by the ultraviolet rays. For this reason, UV sterilizers of this type are operated by an administrator or the like after business hours or the like when human activity has ended, and no people are present.

As is described in Patent Document 1, recent years have seen the on-going development of technology that sterilizes rooms and facilities and the like by tracking people in such places and irradiating ultraviolet rays in such a way as to avoid these people.

### Documents of the Prior Art

### PATENT DOCUMENTS

[Patent Document 1] U.S. Pat. No. 10,639,390

### DISCLOSURE OF THE INVENTION

### Problems to be Solved by the Invention

However, in the case of the former, it is not possible to perform sterilization when people are present and active in a room or a facility. Accordingly, during such periods of human activity, it is not possible to eradicate bacteria and viruses, so that neither is it possible to prevent these from infecting people.

In the case of the latter, because sterilization by means of ultraviolet rays is possible even in areas where people are active, the former problem tends to be avoided. However, because the latter method involves simply irradiating ultraviolet rays in such as way as to avoid people, it is not possible to set locations where intensive sterilization is desired. Accordingly, not only is this method lacking from the standpoint of optimizing the sterilization efficiency, but it is also lacking from the standpoint of ensuring safety in unforeseen circumstances such as if a person suddenly runs into an area being sterilized, and insufficient consideration has been given to dealing with circumstances such as these.

The present invention was conceived in order to make it possible, in a sterilization method and the like that sterilizes a room or the like using sterilization electromagnetic waves including ultraviolet waves, to solve the above-described problems and enable optimal, safe sterilization to be achieved even when people are present.

### Means for Solving the Problem

A sterilization method according to the present invention is characterized in including sterilization area setting in order to set in advance a sterilization area within a predetermined area of human activity, peripheral area setting in order to set in advance peripheral areas that are adjacent to or in proximity to the sterilization area, irradiation mode deciding in order to decide an irradiation mode for irradiating sterilization electromagnetic waves onto the sterilization area based on peripheral area information acquired from the peripheral areas, and sterilization electromagnetic wave irradiating in order to irradiate the sterilization area with sterilization electromagnetic waves in the irradiation mode.

### Effects of the Invention

According to the above-described structure, it is possible by utilizing an autonomous or automatic sterilization mode to implement settings that will enable sterilization to be performed even in areas in which people are active and, in accordance, for example, with a determination made by a user, will enable this sterilization to be concentrated in areas where the sterilization will have a greater effect, and will prevent sterilization from being performed in areas where the sterilization will have little effect. Because of this, it is possible to sterilize, both efficiently and in a shorter period of time, areas having a greater need of being sterilized.

Moreover, when performing autonomous sterilization, because the irradiation mode of the sterilization electromagnetic waves is determined after considering not only information showing, for example, whether or not people are present in the sterilization area, but also information pertaining to peripheral areas thereto, it is possible to improve safety.

### BRIEF DESCRIPTION OF THE DRAWINGS

FIG. 1 is an overall schematic view of a sterilization system according to an embodiment of the present invention.

FIG. 2 is a plan view showing sterilizers and a human activity area according to the same embodiment.

FIG. 3 is a schematic view of a sterilizer according to the same embodiment.

FIG. 4 shows an example of a user dashboard displayed on an information processing terminal used by a user according to the same embodiment.

FIG. 5 is a screen view showing an area setting screen according to the same embodiment.

FIG. 6 is a screen view showing a secondary ultraviolet irradiation area according to the same embodiment.

FIG. 7 is a screen view showing an area setting screen according to the same embodiment.

FIG. 8 is a flowchart showing an operation of a sterilization system according to the same embodiment.

FIG. 9 is a flowchart showing an operation of the sterilization system according to the same embodiment.

### DESCRIPTION OF EMBODIMENTS

Hereinafter, an embodiment of the present invention will be described with reference to the drawings.

### 1. OVERALL STRUCTURE

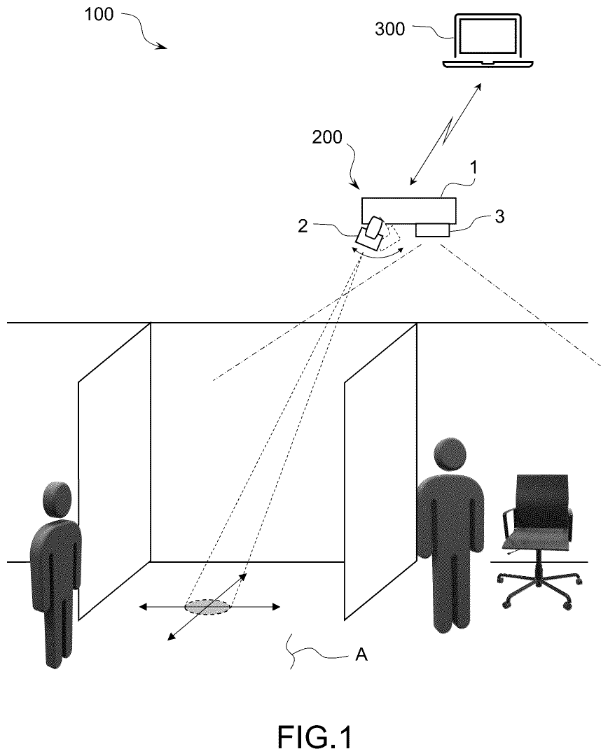

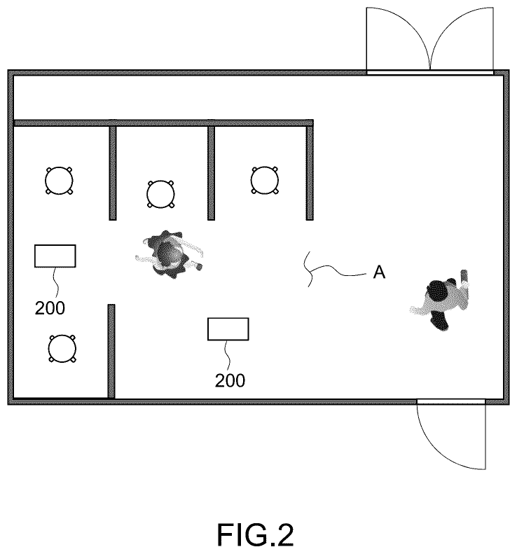

As is shown in FIG. 1 and FIG. 2, a sterilization system 100 according to the present embodiment is provided with either one or a plurality of sterilizers 200 that are installed in a human activity area A such as a room or a facility where people are coming and going, or are remaining present for extended periods, and an information processing terminal 300 that is connected to the sterilizer 200 and is able to communicate therewith. Note that, here, the term ‘sterilization’ refers to eradicating or reducing not only bacteria, but also other (harmful) microbes and viruses.

### 2. STRUCTURE OF EACH PORTION

### (1) Sterilizer 200

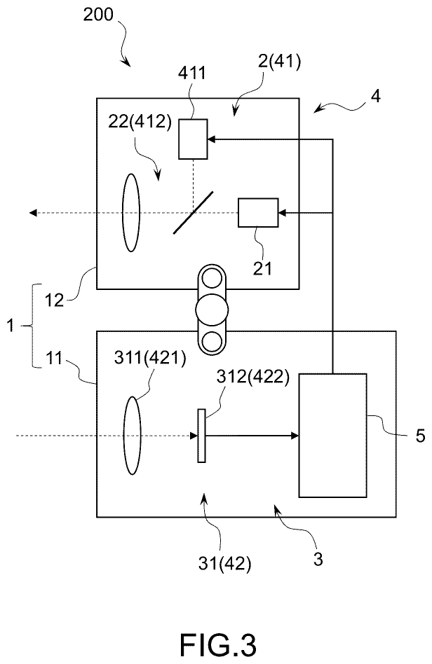

As is shown in FIG. 1 and FIG. 2, each sterilizer 200 is provided with a housing 1 that is mounted on a ceiling, for example, of the human activity area A, an ultraviolet ray irradiation device 2 that is housed within the housing 1, a human recognition sensor 3, an ultraviolet ray irradiation range detection mechanism 4 (shown in FIG. 3), a human motion detector (not shown in the drawings), and a control device 5.

Structures of each of these portions will now be described in detail with reference to FIG. 3.

### (1-1) Housing 1

The housing 1 is provided with a main casing 11, a movable casing 12 that is mounted on the main casing 11 by means of a universal coupling or the like in such a way that an attitude thereof is able to be altered, and a drive mechanism such as a motor or the like (not shown in the drawings) that drives the movable casing 12 so as to alter the attitude thereof.

### (1-2) Ultraviolet Ray Irradiation Device 2 (Sterilization Electromagnetic Wave Irradiation Device)

This ultraviolet ray irradiation device 2 is provided, here, with a first light source body 21 such as an ultraviolet LED or a mercury lamp or the like, and a first projection optical system 22 such as a lens or half-mirror or the like, and emits ultraviolet rays (i.e., sterilization electromagnetic waves, for example, UV-C) having a predetermined directivity from an emission window provided in the movable casing 12 so as to correspond to the attitude direction of this movable casing 12.

### (1-3) Human Recognition Sensor 3

The human recognition sensor 3 detects the presence of a person as well as the position thereof. Here, the human recognition sensor 3 is provided with a visible light camera 31 that is held in the main casing 11, and image analysis software that specifies the position of a person from visible light image data acquired by the visible light camera 31.

The visible light camera 31 is provided with a light-receiving optical system 311 that includes a wide-angle lens and is disposed at a bottom surface of the main casing 11, and a two-dimensional area sensor 312 that detects visible light from the light-receiving optical system 311 that has been guided to and focused on the two-dimensional area sensor 312. The visible light camera 31 is installed facing in a direction and at an attitude that enables it to acquire images of the entire human activity area A.

Here, the image analysis software is installed in the control device 5 (described below). This image analysis software employs AI (Artificial Intelligence) in order to analyze the visible light image data, specify the positions of people in the human activity area A, and output human position data that shows the specified positions. More specifically, this image analysis software is supplied in advance with teaching data in the form of image data for a variety of people, and then performs machine learning of this data in order to be able to determine a person from the visible light image data for the human activity area A and specify the position thereof. The method employed here for this machine learning is assumed to be deep learning, however, the present invention is not limited to this. Note that the image analysis software is not limited to software that utilizes AI, and non-AI software that recognizes people based on differences between image data or on image data that has undergone edge processing, or software that recognizes people using these methods in conjunction with AI may also be used.

### (1-4) Ultraviolet Ray Irradiation Range Detection Mechanism 4 (Sterilization Electromagnetic Wave Irradiation Range Detection Mechanism)

Here, the ultraviolet ray irradiation range detection mechanism 4 is provided with an infrared ray emitting device 41 (i.e., a non-sterilization electromagnetic wave emitting device), and an infrared ray detecting sensor 42 (i.e., a non-sterilization electromagnetic wave detecting sensor).

The infrared ray emitting device 41 is provided with a second light source 411 such as an infrared LED or a halogen lamp or the like, and a second projection optical system 412 such as a lens or mirror or the like, and is formed so as to emit from the emission window of the movable casing 12 infrared rays (i.e., non-sterilization electromagnetic waves, for example, IR) that have equal directivity and are essentially coaxial with the ultraviolet rays, namely, is formed in such a way that an area onto which the infrared rays are irradiated coincides with an area onto which the ultraviolet rays are irradiated. Note that, in the drawings, a portion of the second projection optical system 412, such as the lens and the like, is shared by the first projection optical system 22, however, it is also possible for the second projection optical system 412 to not share any portion of the first projection optical system 22 and to, instead, be formed as a separate body that is located adjacently thereto.

Here, the infrared ray detecting sensor 42 is an infrared camera that acquires images of the human activity area A. The infrared ray detecting sensor 42 is provided with a second light-receiving optical system 421 that includes a wide-angle lens and an infrared ray transmission filter and is disposed at a bottom surface of the main casing 11, and a two-dimensional area sensor 422 that detects infrared rays introduced from the second light-receiving optical system 421. The infrared ray detecting sensor 42 acquires images of the human activity area A, and outputs infrared ray image data showing the area onto which the infrared rays were irradiated.

In this way, as is described above, because the area where the infrared rays are irradiated coincides with the area where the ultraviolet rays are irradiated, the area detected using the infrared ray image data forms the ultraviolet ray irradiation area.

Note also that, in the drawings, the two-dimensional area sensor 422 and the lens of the infrared ray detecting sensor 42 are shared by the visible light camera 31, and it is possible to select whether to acquire visible light images or infrared ray images by switching a bandpass filter (not shown in the drawings), however, it is also possible for the infrared ray detecting sensor (i.e., an infrared ray camera) 42 and the visible light camera 31 to be formed as separate bodies that are located adjacent to each other.

### (1-5) Human Motion Detector

In the present embodiment, a human motion detector (such as a TOF sensor or a pyroelectric sensor (not shown in the drawings)) that is mounted in parallel with the ultraviolet ray emitting device 2 is provided separately from the human recognition sensor 3. The human motion detector detects whether or not a person is present and performs a role of backup to the human recognition sensor 3.

### (1-6) Control Device 5

The control device 5 is a computer that is provided with a CPU, memory, an I/O interface, and a communication interface and the like. As a result of the CPU and the peripheral devices thereof operating in mutual collaboration in accordance with a program stored in the memory, the control device 5 receives signals from the human recognition sensor 3 and the infrared ray detecting sensor 42 and analyzes the contents thereof, and then controls the operations of the ultraviolet ray emitting device 2 and the infrared ray emitting device 41.

### (2) Information Processing Terminal 300

As is shown in FIG. 1, the information processing terminal 300 is used by an administrator of this sterilization system 100 or by an operator or the like (hereinafter, such a person will be referred to by the generic term ‘user’), and is what is known as a general-purpose computer that is provided with a CPU, memory, an I/O interface, a communication interface, a display unit, and input devices such as a mouse and a keyboard or the like. The information processing terminal 300 is connected to the control device 5 so as to be able to communicate therewith via an information communication network such as LAN or WAN, or via a dedicated line or the like.

### 3. SETTINGS AND OPERATION ETC. OF THE STERILIZATION SYSTEM 100

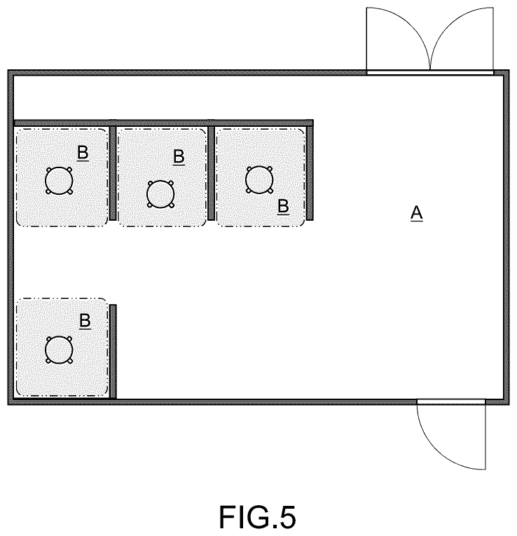

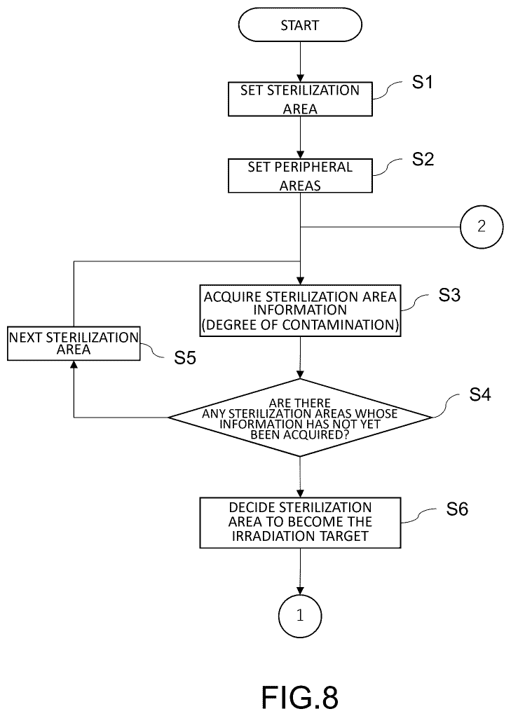

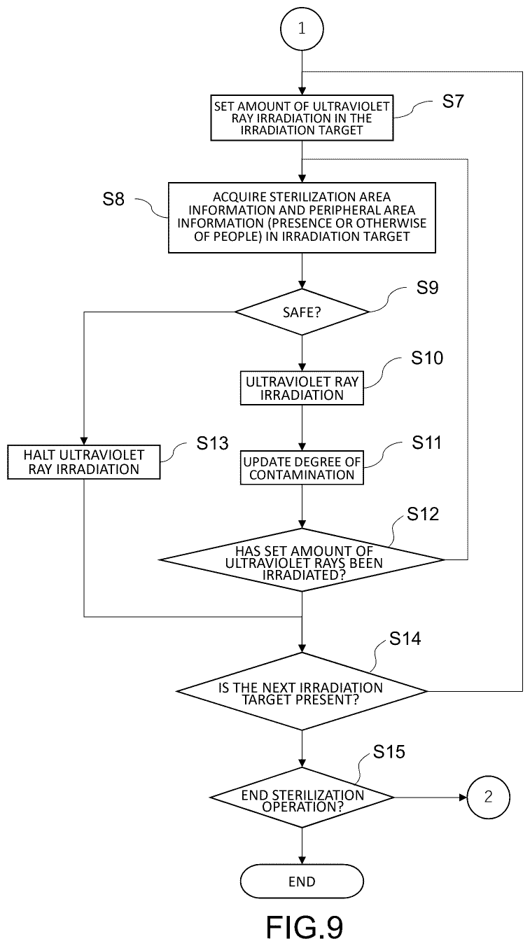

Next, an example of a setting method and operation of the sterilization system 100 will be described with reference to the flowcharts shown in FIG. 8 and FIG. 9, and to the human activity space plan views shown in FIG. 5 through FIG. 7.

### (1) Initial Settings

### (1-1) Setting of Sterilization Area (Step S1 in FIG. 8)

Firstly, a sterilization area is defined by a user, in other words, sterilization area setting is performed.

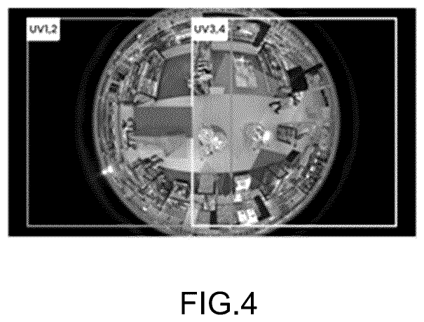

More specifically, the information processing terminal 300 displays a visible light image (an example is shown in FIG. 4) of the human activity area A that was acquired by the visible light camera 31 on the display unit thereof (i.e., on a user dashboard), and requests that the user make an input designating a sterilization area, which is an area which the user wishes to directly irradiate with ultraviolet rays, within the visible light image. Note that, instead of a visible light image, it is also possible to display on the display unit a graphic of the human activity area A such as the CAD drawing shown in FIG. 2. Note also that the description below is for a case in which a graphic such as the one shown in FIG. 2 is displayed on the display unit.

Next, the user makes an input specifying either one or a plurality of sterilization areas B within the human activity area image on the display unit, as is shown in FIG. 5. As a result, the control device 5 receives sterilization area data, which is data showing the respective sterilization areas B, from the information processing terminal 300 and stores this data in a predetermined area of the memory.

Note that, at this time, a 3D layout of the human activity area or of the sterilization areas is detected by the TOF sensor, and an azimuth orientation of each portion of the sterilization areas is calculated by means of an arithmetic calculation performed by the control device 5. As a result, the control device 5 arithmetically calculates an angle between the direction of the irradiated ultraviolet light and the surfaces of each portion of the sterilization area, and uses this information as calibration data in order to secure the amount of ultraviolet ray irradiation that is necessary to achieve sterilization. For example, in a case in which the irradiation angle is shallow, by prolonging the irradiation time in accordance with this shallow angle, it is possible to secure the required amount of irradiation.

### (1-2) Setting of the Peripheral Areas (Step S2 in FIG. 8)

Next, peripheral areas are defined by a user, in other words, peripheral area setting is performed.

More specifically, firstly, in accordance with an instruction from the control device 5, infrared rays are irradiated onto the sterilization areas B from the infrared ray emitting device 41. In a case in which the sterilization area is larger than an area covered by a single infrared irradiation, the infrared rays are scanned and the infrared rays are irradiated so as to thoroughly cover the entire sterilization area.

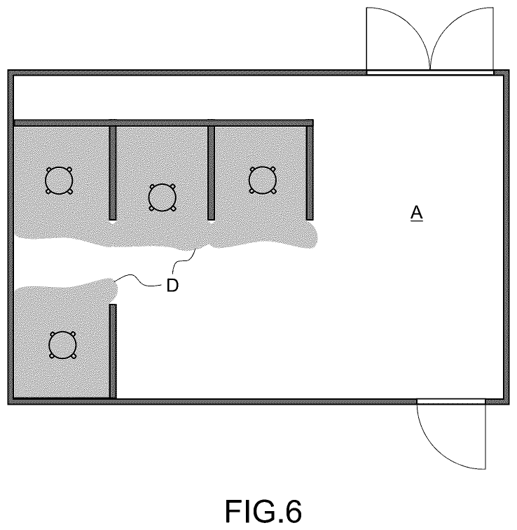

While this infrared ray irradiation is being performed, an infrared ray image of the human activity area A is acquired by the infrared ray camera in accordance with an instruction from the control device 5. As a result, as is shown in FIG. 6, secondary ultraviolet ray irradiation areas D are detected which are areas where not only are the infrared rays irradiated directly onto the sterilization area, but also infrared rays are secondarily irradiated via reflection or scattering or the like and, accordingly, which form the range of impact where there is a strong possibility the ultraviolet rays will be irradiated secondarily. This is impact range acquiring processing and may be considered as a simulation of an ultraviolet ray irradiation.

The control device 5 then transmits secondary ultraviolet ray irradiation area data, which is data showing this secondary ultraviolet ray irradiation area, to the information processing terminal 300.

Upon receiving this data, the information processing terminal 300 displays the secondary ultraviolet ray irradiation areas D in the visible light image being displayed on the display unit, and requests that the user make an input designating peripheral areas C in the areas outside the sterilization areas B.

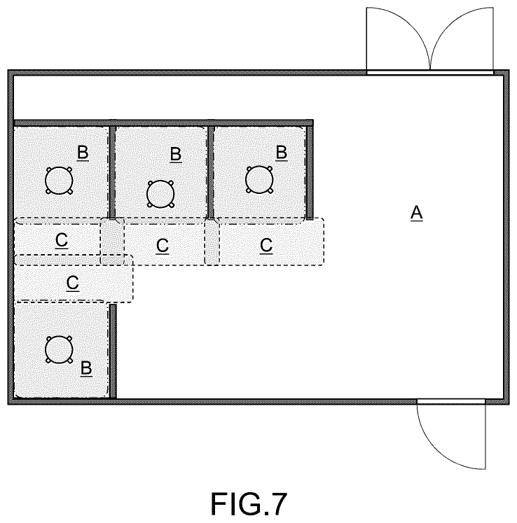

Because the peripheral areas are areas that cannot be considered as safe when irradiated with ultraviolet rays, as is shown in FIG. 7, while referring to the secondary ultraviolet ray irradiation areas D, the user utilizes an input device such as a mouse or a touch panel in order to specify the peripheral areas C that correspond to the respective sterilization areas B (it is likely that the user will specify the peripheral areas C in such a way that they include all of the secondary ultraviolet ray irradiation areas D).

As a result of this, the control device 5 receives peripheral area data, which is data showing the peripheral areas C, from the information processing terminal 300 and stores this data in a predetermined area of the memory.

### (2) Acquiring Sterilization Area Information (Degree of Contamination) (Steps S3˜S5 in FIG. 8)

Next, the control device 5 acquires the degree of contamination, which is one of the sterilization area information items.

After a user has completed the initial settings and begun operating this sterilization device, firstly, human recognition is performed by the human recognition sensor 3.

More specifically, based on the visible light image data, the human recognition sensor 3 specifies the position of a person at regular predetermined sampling times.

The control device 5 sequentially acquires this human position data and, based on this, calculates the position and movement trajectory of a person within the human activity area A. In addition, the control device 5 calculates a degree of contamination which corresponds to the length of stay of the person in each location within the human activity area A, and stores contamination degree data which shows the degree of contamination in each sterilization area in a predetermined area of the memory.

This degree of contamination increases proportionally as the length of stay of the person increases, however, in a case in which the length of time a person is outside the human activity area A continues for a predetermined time or longer, then the degree of contamination may be reduced in accordance with this.

### (3) Setting Irradiation Target (Step S6 in FIG. 8)

In a case in which the degree of contamination in the sterilization area exceeds a set threshold value that has been determined in advance, the control device 5 registers that sterilization area in a predetermined area of the memory as an irradiation target.

### (4) Calculation of Ultraviolet Ray Irradiation Amount (Step S7 in FIG. 9)

Next, the control device 5 selects one irradiation target in accordance with a predetermined sequence from among those irradiation targets (i.e., sterilization areas) that have been confirmed as being safe, and calculates the amount of ultraviolet ray irradiation required for disinfection of that irradiation target in accordance with the degree of contamination therein. At this time, the amount of ultraviolet ray irradiation is calibrated in accordance with the distance from the ultraviolet ray emitting device 2 to the sterilization area, and with the azimuth orientation of the surface being disinfected.

Note that, although the amount of ultraviolet ray irradiation is determined by multiplying the irradiation intensity by the irradiation time, here, because the intensity of the ultraviolet rays is kept constant, in a case in which the irradiation amount is to be increased, then the irradiation time is increased. If the irradiation time is to be kept constant, then the irradiation intensity is increased.

### (5) Safety (i.e., for the Presence of People) Verification (Steps S8, S9 in FIG. 9)

Next, using the human recognition sensor 3, the control device 5 verifies any human presence, which is one of the items of information of the sterilization area registered as the irradiation target and also of the peripheral areas attached to this sterilization area.

At this time, it is also possible to ensure safety by reverifying (i.e., by performing a backup verification) that no persons are present in the irradiation direction using the human motion detector mounted in parallel with the ultraviolet ray emitting device 2.

Furthermore, the control device 5 verifies that the ultraviolet rays are actually being directed towards the target position, and that reflected light or the like therefrom is not escaping to the outside of the peripheral areas by operating the ultraviolet ray irradiation range detection mechanism 4 so as to irradiate infrared rays prior to performing the ultraviolet ray irradiation and then acquiring the resulting infrared images.

Here, if safety cannot be verified, the control device 5 does not perform the ultraviolet ray irradiation of the sterilization area for the time being, but instead moves on to processing the next ultraviolet ray irradiation target (Steps S13, S14 in FIG. 9).

### (6) Ultraviolet Ray Irradiation (Step S10)

Once safety has been verified, the control device 5 controls the ultraviolet ray emitting device 2 so that ultraviolet rays are irradiated onto the sterilization area serving as the target until the amount of irradiation (here, this is the irradiation time) that was calculated in the manner described above is achieved.

The safety confirmation steps S8 and S9 continue to be performed while the ultraviolet rays are being irradiated. If the safety conditions are breached, then the ultraviolet ray irradiation is immediately interrupted. (Step S13 in FIG. 9).

When the ultraviolet ray irradiation is completed or interrupted, the control device 5 moves on to processing the next ultraviolet ray irradiation target (Step S14 in FIG. 9).

Once sterilization has been completed in all of the irradiation targets, provided that a command to end the sterilization operation has not been given (Step S15 in FIG. 9), the control device returns once more to step S3 and starts processing each sterilization area.

### (7) Updating the Degree of Contamination (Step S11 in FIG. 9)

The control device 5 senses the degree of contamination in the relevant sterilization area in accordance with the amount of ultraviolet ray irradiation. More specifically, here, the degree of contamination is reduced in accordance with the irradiation time. Naturally, the degree of contamination at the point in time when the ultraviolet ray irradiation ends normally is a lower value than the set threshold value.

In contrast, in a case in which, for some reason or other, the ultraviolet ray irradiation is interrupted, the degree of contamination at that point in time is held in the memory. In a case in which the degree of contamination still exceeds the set threshold value, the registration of that sterilization area as an irradiation target is continued, and ultraviolet ray irradiation is commenced once again when the safety conditions have been restored.

### (8) Display of Sterilization Situation

The control device 5 sequentially transmits situation data that shows the sterilization situation of the human activity area A to the information processing terminal. The information processing terminal 300 is formed such that, either in response to an operation performed by a user, or as a normal state of affairs, the sterilization situation is able to be displayed on the display unit.

Here, the term ‘sterilization situation’ refers to the sterilization processing situation in each sterilization area B (i.e., whether each sterilization area B is awaiting disinfection, or is currently being disinfected, or has completed disinfection), and indicates the degree of contamination and the like of each sterilization area B. Using this, a user is able to verify the situation in each sterilization area and select sterilization areas B that are able to be used.

### 4. EFFECTS

According to the above-described structure, because it is possible, using a completely autonomous or automatic sterilization system that employs AI or the like, to implement settings that will enable sterilization to be performed even during periods when people are active, and, without relinquishing control solely to AI, but by also relying on the judgement of a user, to concentrate sterilization in areas where the sterilization will be most effective, and to not perform sterilization in areas where the sterilization will have the least effect, it is possible to efficiently sterilize areas where sterilization is absolutely required in a short period of time, and to design and carry out a procedure that fully reflects the intention of the user.

Moreover, when performing autonomous sterilization, because the mode of irradiation of sterilization electromagnetic waves is determined after considering not only information showing, for example, whether or not a person is present in a sterilization area, but also information pertaining to peripheral areas thereto, it is possible to improve safety markedly.

### 5. CHARACTERISTICS OF THE STERILIZATION SYSTEM 100

A summary of the characteristics of the above-described sterilization system 100 will now be given.

### (Disclosure 1)

A sterilization method for sterilizing a predetermined area of human activity A characterized in that sterilization area setting is performed in order to set in advance a sterilization area within the area of human activity A, peripheral area setting is performed in order to set in advance peripheral areas that are adjacent to or in proximity to the sterilization area, peripheral area information acquiring is performed in order to acquire peripheral area information, which is information relating to the peripheral areas, irradiation mode deciding is performed in order to decide an irradiation mode for irradiating ultraviolet rays (i.e., sterilization electromagnetic waves) onto the sterilization area based on the peripheral area information, and ultraviolet ray (i.e., sterilization electromagnetic wave) irradiation is performed in order to irradiate ultraviolet rays (i.e., sterilization electromagnetic waves) onto the sterilization area in the irradiation mode.

### (Disclosure 2)

The sterilization method described in Disclosure 1 characterized in that impact range acquiring is additionally performed in order to acquire an impact range which is a range likely to be affected by the sterilization electromagnetic waves, and the peripheral areas are set based on the impact range, and the impact range is detected or arithmetically calculated by irradiating non-sterilization electromagnetic waves onto the sterilization area.

### (Disclosure 3)

The sterilization method described in Disclosure 1 or 2 characterized in that, in the peripheral area setting, impact range displaying is additionally performed in order to display the impact range on a display unit.

### (Disclosure 4)

The sterilization method described in any one of Disclosures 1 through 3 characterized in that human presence information showing whether or not a person is present within the peripheral areas is included in the peripheral area information, and in a case in which the human presence information shows that a person is present within the peripheral areas, an irradiation intensity, which is one of the irradiation modes of the ultraviolet rays (i.e., of the sterilization electromagnetic waves), is either reduced compared to a case in which the human presence information shows that a person is not present within the peripheral areas, or is set to 0.

### (Disclosure 5)

The sterilization method described in any one of Disclosures 1 through 4 characterized in that the irradiation mode is decided based additionally on sterilization area information acquired from the sterilization area, and a degree of contamination of the sterilization area and human presence information are included in the sterilization area information, and in a case in which a person is present in the sterilization area, the irradiating of the ultraviolet rays (i.e., the sterilization electromagnetic waves) is halted and an irradiation amount, an irradiation intensity, or an irradiation location, which are irradiation modes for irradiating the sterilization electromagnetic waves onto the relevant sterilization area after the person has departed therefrom, or else a desired combination of these are decided in accordance with the degree of contamination in the relevant sterilization area prior to the ultraviolet rays being irradiated thereon.

### (Disclosure 6)

The sterilization method described in Disclosure 5 characterized in that the degree of contamination is determined by the length of stay of a person prior to sterilization.

### (Disclosure 7)

A sterilization system characterized in being provided with an ultraviolet ray (i.e., a sterilization electromagnetic wave) emitting device 2 that emits ultraviolet rays (i.e., sterilization electromagnetic waves), and a control device 5 that controls the ultraviolet ray (i.e., sterilization electromagnetic wave) emitting device 2, and in that the control device 5 sets a sterilization area within an area of human activity A based on an input from a user, sets peripheral areas that are adjacent to or in proximity to the sterilization area, acquires peripheral area information, which is information relating to the peripheral areas, and, based on the peripheral area information, decides an irradiation mode for irradiating the ultraviolet rays (i.e., sterilization electromagnetic waves) onto the sterilization area, and then controls the ultraviolet ray (i.e., sterilization electromagnetic wave) emitting device 2 such that this irradiation mode is implemented.

### 6. ADDITIONAL EMBODIMENTS

The present invention is not limited to the above-described embodiment.

In the above-described embodiment, the peripheral areas are set via an input from a user, however, it is also possible for the control device to set peripheral areas automatically based on detected secondary ultraviolet ray irradiation areas such that these are included in the peripheral areas.

In the above-described embodiment, the peripheral area information only indicated whether a person was present or not, however, it is also possible for the peripheral area information to include, for example, the direction of movement and/or the movement speed of a person. For example, if a person is moving in a direction that will take them out of the sterilization area, then ultraviolet ray irradiation or the like can be performed even if a person is present within a peripheral area.

In the above-described embodiment, the length of stay of a person in a sterilization area and the presence or otherwise or a person in a sterilization area are given as examples of sterilization area information, however, it is also possible for the sterilization area information to include the length of time a person was continuously not present in a sterilization area. For example, if the length of time that a person was not present in a sterilization area is prolonged, then it is possible for the degree of contamination to be lowered.

In the above-described embodiment, in a case in which a person is present in a peripheral area, the ultraviolet ray irradiation in the corresponding sterilization area is halted, however, it is also possible to simply lower the irradiation intensity thereof instead of reducing it to zero. Moreover, in a case in which a person has not actually entered a sterilization area, but has simply walked past it, then the irradiation intensity can be restored to its previous level so that sterilization of the sterilization area is restarted.

In the above-described embodiment, the length of stay of a person in a sterilization area is scored as a degree of contamination, and the sterilization intensity for that sterilization area is determined based on the value of this score, however, it is also possible to leave the degree of contamination as undetermined and to perform a uniform sterilization. In this case, consideration may be given to performing sterilization in sequence starting from the sterilization area that has been sterilized the fewest number of times during a fixed period in the past.

The sterilization electromagnetic waves are not limited to ultraviolet rays and other electromagnetic waves such as blue light or microwaves or the like may also be used. In addition, various modifications and the like may be made to the present invention insofar as they do not depart from the spirit or scope of the present invention. For example, other types of devices and sensors than those described above may be used as the respective devices and sensors of the above-described embodiment provided that they are able to perform the same functions.

What is claimed is:

1. A sterilization method for sterilizing a predetermined area of human activity comprising:
setting sterilization area within the area of human activity;
setting peripheral area that are adjacent to or in proximity to the sterilization area;
acquiring peripheral area information relating to the peripheral area;
determining an irradiation mode for irradiating sterilization electromagnetic waves onto the sterilization area based on the peripheral area information; and
irradiating sterilization electromagnetic wave onto the sterilization area in the irradiation mode.

2. The sterilization method according to claim 1, wherein
impact range acquiring is additionally performed in order to acquire an impact range which is a range likely to be affected by the sterilization electromagnetic waves, and
the peripheral areas are set based on the impact range, and
the impact range is detected or arithmetically calculated by irradiating non-sterilization electromagnetic waves onto the sterilization area.

3. The sterilization method according to claim 2, wherein, in the peripheral area setting, impact range displaying is additionally performed in order to display the impact range on a display device.

4. The sterilization method according to claim 1, wherein human presence information showing whether or not a person is present within the peripheral areas is included in the peripheral area information, and in a case in which the human presence information shows that a person is present within the peripheral areas, an irradiation intensity, which is one of the irradiation modes of the sterilization electromagnetic waves, is either reduced compared to a case in which the human presence information shows that a person is not present within the peripheral areas, or is set to 0.

5. The sterilization method according to claim 1, wherein the irradiation mode is decided based additionally on sterilization area information acquired from the sterilization area, and
a degree of contamination of the sterilization area and human presence information are included in the sterilization area information, and
in a case in which a person is present in the sterilization area, the irradiation of the sterilization electromagnetic waves is halted and an irradiation amount, an irradiation intensity, or an irradiation location, which are irradiation modes for irradiating the sterilization electromagnetic waves onto the relevant sterilization area after the person has departed therefrom, or else a desired combination of these are decided in accordance with the degree of contamination in the relevant sterilization area prior to the sterilization electromagnetic waves being irradiated thereon.

6. The sterilization method according to claim 5, wherein the degree of contamination is determined by the length of stay of a person prior to sterilization.

7. A sterilization system that sterilizes a predetermined area of human activity comprising:
a sterilization electromagnetic wave emitting device that emits sterilization electromagnetic waves; and
a control device that controls the sterilization electromagnetic wave emitting device, wherein the control device:
sets a sterilization area within the area of human activity based on an input from a user;
sets peripheral areas that are adjacent to or in proximity to the sterilization area;
acquires peripheral area information, which is information relating to the peripheral areas; and,
based on the peripheral area information, decides an irradiation mode for irradiating the sterilization electromagnetic waves onto the sterilization area, and then controls the sterilization electromagnetic wave emitting device such that this irradiation mode is implemented.
